(19) United States
(12) Patent Application Publication
(10) Pub. No.: US 2015/0174528 A1
(43) Pub. Date: Jun. 25, 2015

(54) APPARATUS FOR CLEANING FLUID

(71) Applicant: Seoul Viosys Co., Ltd., Ansan-si (KR)

(72) Inventors: Young Hwan Son, Ansan-si (KR); Daewoong Suh, Ansan-si (KR); Seong Min Lee, Ansan-si (KR)

(21) Appl. No.: 14/411,684

(22) PCT Filed: Jun. 27, 2013
(86) PCT No.: PCT/KR2013/005674

§ 371 Date: Dec. 29, 2014

(30) Foreign Application Priority Data
Jun. 29, 2012 (KR) 10-2012-0071299

(51) Int. Cl. B01D 53/88; A61L 2/08; A61L 9/20; B01D 53/86

(52) U.S. Cl. CPC B01D 53/885; B01D 53/8678; A61L 2/088; A61L 9/205; B01D 2255/802; B01D 2255/40; B01D 2255/20707; B01D 2255/20776; B01D 2255/20792; B01D 2258/06; B01D 2257/91; B01D 2257/708; B01D 2259/804

(57) ABSTRACT

Provided is an apparatus for cleaning a fluid using an ultraviolet light-emitting diode. The apparatus includes an outer case, a fluid cleaning filter disposed in the outer case and filters the fluid introduced, a photocatalyst coated on a surface of the fluid cleaning filter, and a first light source unit mounted on the outer case to face a rear surface of the fluid cleaning filter in a direction in which the fluid is introduced, and emits light having a plurality of wavelength bands toward the rear surface of the fluid cleaning filter. Since the apparatus may have high sterilization and deodorization effects by emitting light having a plurality of wavelength bands and may improve filter reuse efficiency using a filter on which a photocatalyst is coated, the filter may be semi-permanently used and fluid cleaning efficiency may be improved.

### TECHNICAL FIELD

The present invention relates to an apparatus for cleaning a fluid, and more particularly, to an apparatus for cleaning a fluid using an ultraviolet light-emitting diode.

### BACKGROUND ART

Nowadays, most people live in cities, and most cities are contaminated with various contaminants discharged from automobiles, industries, etc. In particular, contaminants included in yellow dust blown from China pass over the Pacific Ocean and reach as far as the western part of the U.S. Since the contaminants may cause various diseases as well as respiratory ailments such as a bronchial trouble, it is necessary to remove the contaminants.

A representative method from among methods of removing such contaminants in the air is an air cleaning filter method using an air cleaning filter. The filter method has a problem in that since the air cleaning filter has to be periodically replaced, maintenance costs are continuously incurred. Also, the filter method has problems in that when cleaning is not periodically performed or the air cleaning filter is not periodically replaced, bacteria may propagate in the air cleaning filter and the contaminants may be ejected, thereby rather degrading indoor air quality.

To solve these problems, a technology of removing bacteria, mold, etc. growing in a filter by emitting light energy such as ultraviolet light to the filter has been developed. Such a method of sterilizing a filter using an ultraviolet lamp or the like is effective in removing bacteria and mold in the filter but has a problem in that foreign materials, dust, etc. gather on a surface of the lamp during use, thereby reducing light emission efficiency. Also, the method has a problem in that due to a space occupied by the ultraviolet lamp or the like, a volume of an apparatus is increased, thereby limiting space utilization.

Accordingly, there is a demand for an air cleaning apparatus that may semi-permanently use a filter using high sterilization efficiency of ultraviolet light and may maintain high light emission efficiency of a light source.

### DISCLOSURE OF INVENTION

### Technical Problem

The present invention is directed to providing an apparatus for cleaning a fluid which may enable semi-permanent use of a filter using high sterilization efficiency of ultraviolet light and maintain high light emission efficiency of a light source.

### Solution to Problem

One aspect of the present invention provides an apparatus for cleaning a fluid, the apparatus including: an outer case; a fluid cleaning filter disposed in the outer case and filters the fluid introduced; a photocatalyst coated on a surface of the fluid cleaning filter; and a light source unit mounted on the outer case to face a rear surface of the fluid cleaning filter in a direction in which the fluid is introduced, and emits light having a plurality of wavelength bands toward the rear surface of the fluid cleaning filter.

Another aspect of the present invention provides an apparatus for cleaning a fluid, the apparatus including: an outer case; a fluid cleaning filter mounted on an end of the outer case and filters the fluid introduced; and a light source unit disposed along a circumference of the outer case close to a front surface of the fluid cleaning filter in a direction in which the fluid is introduced, and emits light having a plurality of wavelength bands toward the fluid introduced toward the fluid cleaning filter.

### Advantageous Effects of Invention

Since an apparatus for cleaning a fluid of the present invention may have high sterilization and deodorization effects by emitting light having a plurality of wavelength bands and may improve filter reuse efficiency using a filter on which a photocatalyst is coated, the filter may be semi-permanently used and fluid cleaning efficiency may be improved.

However, technical effects of the present invention are not limited thereto, and other unmentioned technical effects will be apparent to one of ordinary skill in the art from the following description.

### BRIEF DESCRIPTION OF DRAWINGS

FIG. 1 is a perspective view illustrating an apparatus for cleaning a fluid, according to a first embodiment of the present invention.

FIG. 2 is an exploded perspective view illustrating the apparatus according to the first embodiment of the present invention.

FIG. 3 is a perspective view illustrating an apparatus for cleaning a fluid, according to a second embodiment of the present invention.

FIG. 4 is an exploded perspective view illustrating the apparatus according to the second embodiment of the present invention.

FIG. 5 is a cross-sectional view illustrating an outer case in which a partition wall unit is formed in a direction in which the fluid is introduced.

FIG. 6 is a plan view illustrating a fluid cleaning filter 200 formed of a metal foam 205 and an enlarged view illustrating a portion of the fluid cleaning filter.

### MODE FOR THE INVENTION

Hereinafter, exemplary embodiments of the present invention will be described in detail. However, the present invention is not limited to the exemplary embodiments disclosed below, but can be implemented in various forms. The following exemplary embodiments are described in order to enable those of ordinary skill in the art to embody and practice the invention.

It will also be understood that when a layer is referred to as being “on” another layer or a substrate, it can be directly on the other layer or the other substrate, or intervening layers may also be present therebetween. It will be understood that when the expressions “over,” “on,” and “top surface” may also encompass orientations of “under,” “beneath,” and “bottom surface”. That is, a spatial direction is construed as a relative direction, instead of an absolute direction.

It will be understood that, although the terms “first,” “second,” or “third” may be used herein to describe various elements, these elements should not be limited by these terms. These terms are only used to distinguish one element from another.

Also, in the drawings, thicknesses of layers and regions are exaggerated for clarity.

The same reference numerals denote the same elements throughout.

### First Embodiment

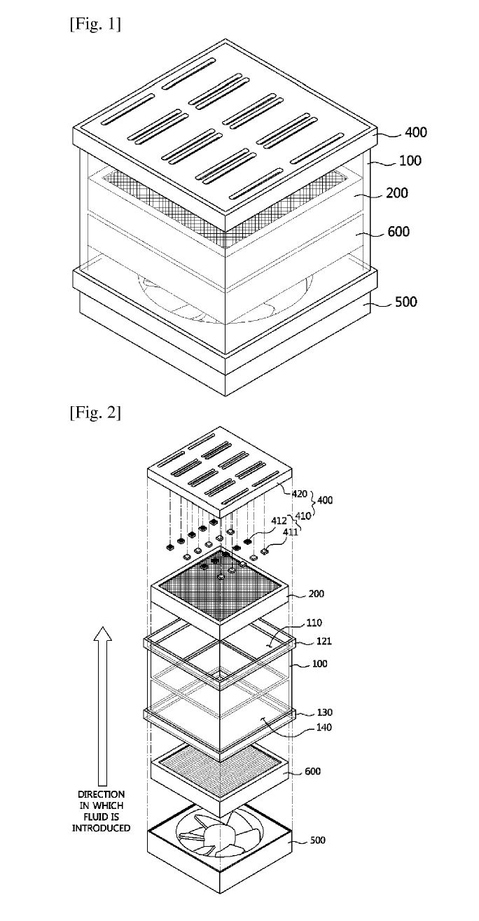

FIGS. 1 and 2 are a perspective view and an exploded perspective view illustrating an apparatus for cleaning a fluid, according to a first embodiment of the present invention, respectively.

Referring to FIGS. 1 and 2, the apparatus according to the first embodiment includes an outer case 100, a fluid cleaning filter 200, and a first light source unit 400. As shown in FIG. 1, the fluid cleaning filter 200 is received in the outer case 100, and filters the fluid introduced into the outer case 100. A photocatalyst 300 is coated on a surface of the fluid cleaning filter 200. The first light source unit 400 emits light toward a photocatalyst-coated surface of the fluid cleaning filter 200. The light emitted by the first light source unit 400 toward the photocatalyst-coated surface of the fluid cleaning filter 200 activates the photocatalyst 300 coated on the surface of the fluid cleaning filter 200, and contaminants adsorbed onto the fluid cleaning filter 200 are decomposed due to the photocatalytic reaction. That is, when the fluid is filtered, the apparatus removes the contaminants adsorbed onto the fluid cleaning filter 200 using the photocatalytic reaction.

The outer case 100 is a space where the fluid to be cleared, that is, the fluid including the contaminants, is introduced and cleared. In the outer case 100, a filter receiving unit 110 in which the fluid cleaning filter 200 may be received and a first light source mounting unit 121 having one end on which the first light source unit 400 may be mounted are formed. Also, according to embodiments, in the outer case 100, a fan mounting unit 130 disposed on the other end of the first light source mounting unit 121 and on which a fan 500 may be mounted, and a pre-filter receiving unit 140 in which a pre-filter 600 may be received are formed.

The fluid cleaning filter 200 crosses an inside of the outer case 100 such that a filtering surface is perpendicular to a direction in which the fluid is introduced. Accordingly, the contaminants such as mites, viruses, mold, bacteria, dust, fumes, volatile organic compounds, or other offensive odor substances included in the fluid introduced into the outer case 100 are adsorbed onto the fluid cleaning filter 200.

The fluid cleaning filter 200 may be formed to have any of various shapes including a honeycomb shape to increase a contact time and a contact area between the fluid and the fluid cleaning filter 200. Also, the fluid cleaning filter 200 may be any of well-known filters such as an activated carbon filter or a high-efficiency particulate air (HEPA) filter, and may be appropriately selected by one of ordinary skill in the art according to properties of the fluid to be cleaned. For example, when the fluid to be cleaned mainly includes fumes, volatile organic compounds, or other offensive odor substances, the fluid cleaning filter 200 may be an activated carbon filter. When the fluid to be cleaned mainly includes mites, viruses, mold, or bacteria, the fluid cleaning filter 200 may be a HEPA filter. Also, the fluid cleaning filter 200 may include one filter, or a plurality of filters. In particular, when the fluid cleaning filter 200 includes a plurality of filters, the plurality of filters may be the same or different kinds of filters.

Also, the fluid cleaning filter 200 may be a metal filter formed of a metal, and more preferably, may be formed of a metal foam 205.

FIG. 6 is a plan view illustrating the fluid cleaning filter 200 formed of the metal foam 205 and an enlarged view illustrating a portion of the fluid cleaning filter 200. As shown in FIG. 6, the metal foam 205 refers to a porous metal structure having a three-dimensional (3D) foam shape, and may be formed of a metal such as nickel, iron, chromium, or aluminum. In particular, the metal foam 205 has advantages in that a surface ratio per unit volume is high due to open pores and 3D cavities therein, it is easy to clean the metal foam 205, and the metal foam 205 may be semi-permanently used.

The photocatalyst 300 is coated on a surface of the fluid cleaning filter 200, that is, a front surface or a rear surface of the fluid cleaning filter, in the direction in which the fluid is introduced. In particular, when the fluid cleaning filter 200 is formed of the metal foam 205, the photocatalyst 300 may be coated on surfaces of all of the open pores and the 3D cavities of the metal foam 205 as shown in FIG. 6. The photocatalyst 300 is activated by light to accelerate decomposition of the contaminants adsorbed onto the fluid cleaning filter 200. The photocatalyst 300 may be any one or more selected from the group consisting of TiO₂, SrTiO₃, WO₃, ZnO, and a mixture thereof. For example, the photocatalyst 300 may be TiO₂.

When light with energy greater than band gap energy of the photocatalyst is emitted, electrons and holes are generated on the surface of the photocatalyst 300. The generated electrons and holes react with moisture and oxygen molecules in the air to generate hydroxyl radicals (OH—) and superoxide anion radicals (O₂—). In this case, the generated hydroxyl radicals are very unstable and are likely to react with other materials. In particular, the hydroxyl radicals have high oxidizing power, and thus oxidize and decompose organic materials including various disease-causing germs and bacteria. For example, when the photocatalyst 300 is TiO₂, the contaminants adsorbed onto the fluid cleaning filter 200 are an ethylene gas (C₂H₄), the ethylene gas is photooxidative-decomposed according to Chemical Formula 1 in moisture of the air, and is removed by the fluid cleaning filter 200.

C₂H₄+4H₂O→2CO₂+6H₂ [Chemical Formula 1]

The first light source unit 400 includes a light-emitting unit 410 and a fixing unit 420, and is mounted on the outer case 100 to emit light toward the photocatalyst-coated surface of the fluid cleaning filter 200. For example, the first light source unit 400 may be mounted on the first light source mounting unit 121 of the outer case 100 to face the front surface or the rear surface of the fluid cleaning filter 200 in the direction in which the fluid is introduced. The first light source unit 400 mounted as described above decomposes the contaminants adsorbed onto the fluid cleaning filter 200 by emitting light to the front surface or the rear surface of the fluid cleaning filter 200, and secondly cleans the fluid that has been cleaned by passing through the fluid cleaning filter 200 by emitting light to the fluid. The first light source unit 400 may be disposed to be spaced apart by about 5 mm to 50 mm, more preferably, about 10 mm to 30 mm, from the fluid cleaning filter 200 so that the entire surface of the fluid cleaning filter 200 is directly exposed to light generated by the first light source unit 400.

The first light source unit 400 may include a light-emitting diode. Light emitted by the light-emitting diode may be ultraviolet light in a wavelength range from 200 nm to 400 nm. Also, the light-emitting diode may emit ultraviolet light having a narrow wavelength width.

The first light-emitting unit 410 emits light having a plurality of wavelength bands. Accordingly, the light-emitting unit 410 may include a first light-emitting diode group 411 that emits light having a first wavelength band and a second light-emitting diode group 412 that emits light having a second wavelength band.

The light having the first wavelength band may be near ultraviolet light having a wavelength band ranging from 350 nm to 400 nm. More preferably, the light having the first wavelength band may be light having a wavelength band ranging from 370 nm to 390 nm. Accordingly, the first light-emitting diode group 411 may include one or more light-emitting diodes emitting light having a wavelength band ranging from 350 nm to 400 nm, more preferably, light having a wavelength band ranging from 370 nm to 390 nm. Light emitted by the first light-emitting diode group 411 is emitted to the rear surface of the fluid cleaning filter 200 to activate the photocatalyst 300 coated on the surface of the fluid cleaning filter 200. Due to the activation of the photocatalyst 300, the contaminants such as organic materials including various disease-causing germs and bacteria adsorbed onto the fluid cleaning filter 200 are photooxidative-decomposed. Once the contaminants adsorbed onto the fluid cleaning filter 200 are decomposed as described above, the fluid cleaning filter 200 may be reused.

The light having the second wavelength band may be deep ultraviolet light having a wavelength band ranging from 200 nm to 300 nm. More preferably, the light having the second wavelength band may be light having a wavelength band ranging from 250 nm to 290 nm. Accordingly, the second light-emitting diode group 412 may include one or more light-emitting diodes emitting light having a wavelength band ranging from 200 nm to 300 nm, more preferably, light having a wavelength band ranging from 250 nm to 290 nm. The light having the second wavelength band emitted by the second light-emitting diode group 412 may be emitted to the photocatalyst 300 to accelerate the photooxidative decomposition, like the light having the first wavelength band. At the same time, the light having the second wavelength band destroys cell walls or cell membranes of microorganisms such as various disease-causing germs and bacteria. Accordingly, the light having the second wavelength band may remove microorganisms included in the fluid through sterilization, without being subjected to the photooxidative decomposition due to the photocatalyst. That is, the light having the second wavelength band may simultaneously perform sterilization and acceleration of activation of the photocatalyst.

As described above, since the light-emitting unit 410 includes not only the first light-emitting diode group 411 but also the second light-emitting diode group 412, photocatalyst activation efficiency and sterilization efficiency of the light source unit 400 may be improved.

The apparatus may further include a fan 500. The fan 500 is mounted on the fan mounting unit 130 of the outer case 100, and functions to suck the fluid into the outer case 100 using a rotational force and to eject cleaned air to the outside. Accordingly, the fan 500 may be disposed to face the front surface or the rear surface of the fluid cleaning filter 200 in the direction in which the fluid is introduced. When the fan 500 is disposed in front of the fluid cleaning filter 200 in the direction in which the fluid is introduced, a rotation surface faces the front surface of the fluid cleaning filter 200. Accordingly, the fluid is sucked into the outer case 100 when the fan 500 rotates, and the fluid sucked into the outer case 100 is cleaned while passing through the fluid cleaning filter 200. In particular, the flow of the fluid sucked into the outer case 100 may be adjusted by adjusting a rotational speed of the fan 500. A contact time between the filter and the fluid or the penetration of the fluid may be adjusted by adjusting the flow of the fluid as described above. Also, a suction force generated toward the inside of the outer case 100 due to the rotation of the fan 500 functions as a driving force that ejects the fluid cleaned while passing through the fluid cleaning filter 200 to the outside, and removes the contaminants decomposed by the photocatalyst 300 on the fluid cleaning filter 200. Also, when the fan 500 is disposed behind the fluid cleaning filter 200 in the direction in which the fluid is introduced, the fan 500 may reduce pressure loss in the flow of the fluid which may occur due to the fluid cleaning filter 200, thereby enabling the fluid to more smoothly flow.

The apparatus may further include a pre-filter 600. The pre-filter 600 is received in the pre-filter receiving unit 140 of the outer case 100, and functions to filter particles having relatively large sizes before the fluid sucked into the outer case 100 due to the fan 500 passes through the fluid cleaning filter 200. Accordingly, the pre-filter 600 may be disposed between the fan 500 and the fluid cleaning filter 200, and may be received in the outer case 100 such that the filtering surface faces the front surface of the fluid cleaning filter 200. For example, the pre-filter 600 may be formed of a nonwoven fabric having fine holes.

### Second Embodiment

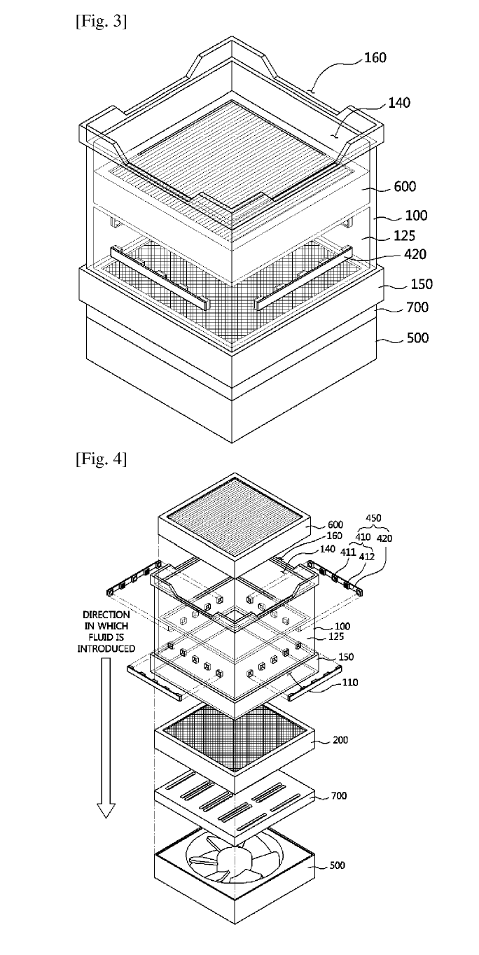

FIGS. 3 and 4 are a perspective view and an exploded perspective view illustrating an apparatus for cleaning a fluid, according to a second embodiment of the present invention, respectively.

The apparatus according to the second embodiment of FIGS. 3 and 4 may be embodied as a separate apparatus for cleaning a fluid from the apparatus of the first embodiment, or may be embodied by adding a structure of a second light source unit 450 to the apparatus of the first embodiment. The following explanation will be given under the assumption that the apparatus is embodied as a separate apparatus from the apparatus of the first embodiment, but the scope of the second embodiment is not limited thereto.

Referring to FIGS. 3 and 4, the apparatus according to the second embodiment includes the outer case 100, the fluid cleaning filter 200, and a second light source unit 450. The outer case 100 is a space where the fluid to be cleaned, that is, the fluid including the contaminants are introduced. A second light source mounting unit 125 on which the second light source unit 450 may be mounted and a filter mounting unit 150 on which the fluid cleaning filter 200 may be mounted are sequentially formed on one end of the outer case 100. In particular, the second light source mounting unit 125 is formed such that the second light source unit 450 is disposed along a circumference of the outer case 100. Also, according to embodiments, the pre-filter receiving unit 140 in which the pre-filter 600 may be received may be additionally formed in the outer case 100, and grooves 160 for facilitating an introduction of the fluid to be cleaned existing along a circumference of the apparatus may be additionally formed along a circumference of the other end of the outer case 100 in the direction in which the fluid to be cleaned is introduced.

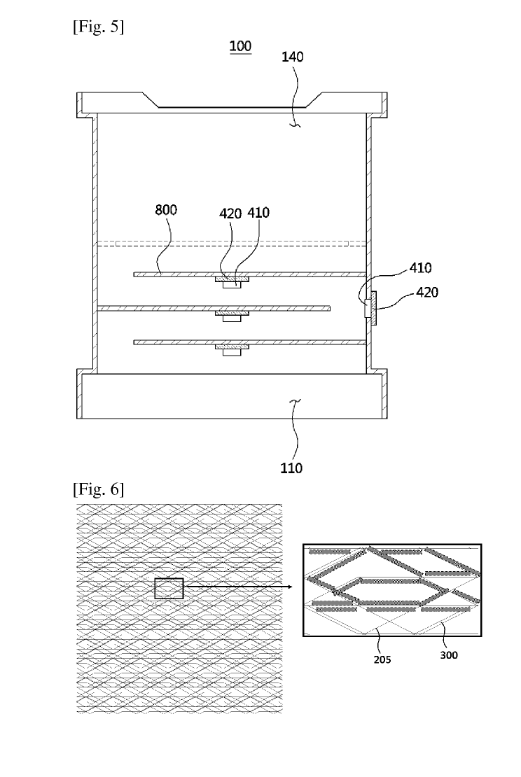

FIG. 5 is a cross-sectional view illustrating the outer case 100 in which a partition wall unit 800 is formed in the direction in which the fluid is introduced.

Referring to FIG. 5, at least one partition wall unit 800 may be formed in the outer case 100. The partition wall unit 800 may be disposed in the outer case to face the front surface of the fluid cleaning filter 200 in the direction in which the fluid is introduced. The partition wall unit 800 protrudes from one wall surface of the outer case 100 to face the front surface of the fluid cleaning filter 200 and divides the inside of the outer case 100 into regions so that the fluid to be cleaned introduced into the outer case 100 flows in the regions defined by the partition wall unit 800. That is, since the fluid introduced into the outer case 100 may flow only in a space formed by the partition wall unit 800 and the outer case 100, the fluid flows along the partition wall unit 800 in the outer case 100. Accordingly, a time taken for the fluid introduced into the outer case 100 to stay in the outer case 100 is further increased due to the partition wall unit 800. When a plurality of the partition wall units 800 are provided, the partition wall units 800 may be disposed to divide the inside of the outer case 100 in a zigzag pattern.

Referring back to FIGS. 3 and 4, the fluid cleaning filter 200 is disposed to cross the inside of the outer case 100 such that the filtering surface is perpendicular to the direction in which the fluid is introduced. Accordingly, the contaminants such as mites, viruses, mold, bacteria, dust, fumes, volatile organic materials, or other offensive odor substances included in the fluid introduced into the outer case 100 are adsorbed onto the fluid cleaning filter 200. A type and a structure of the fluid cleaning filter 200 are the same as those described with respect to the apparatus of the first embodiment.

The photocatalyst 300 may be coated on the surface of the fluid cleaning filter 200, that is, the front surface or the rear surface of the fluid cleaning filter, in the direction in which the fluid is introduced. A type and a function of the photocatalyst 300 are the same as those described with respect to the apparatus of the first embodiment.

The second light source unit 450 includes the light-emitting unit 410 and the fixing unit 420. The light-emitting unit 410 is fixed to the fixing unit 420 and is mounted on the second light source mounting unit 125 of the outer case 100. Accordingly, the second light source unit 450 is disposed along the circumference of the outer case 100, and emits light toward the fluid to be cleaned introduced into the outer case 100. Part of light generated by the second light source unit 450 may be emitted toward the photocatalyst 300 of the fluid cleaning filter 200.

Also, as shown in FIG. 5, when the partition wall unit 800 is additionally formed in the outer case 100, the second light source unit 450 may be disposed on a front surface or a rear surface of the partition wall unit in the direction in which the fluid is introduced. As described above, when the second light source unit 450 is disposed on the front surface or the rear surface of the partition wall unit 800, the second light source unit 450 may emit light toward the fluid while the fluid moves along the space defined by the partition wall unit 800.

The light-emitting unit 410 emits light having a plurality of wavelength bands. Accordingly, the light-emitting unit 410 may include the first light-emitting diode group 411 that emits light having a first wavelength band and the second light-emitting diode group 412 that emits light having a second wavelength band.

The light having the first wavelength band may be near ultraviolet light having a wavelength band ranging from 350 nm to 400 nm. More preferably, the light having the first wavelength band may be light having a wavelength band ranging from 370 nm to 390 nm. Accordingly, the first light-emitting diode group 411 may include one or more light-emitting diodes emitting light having a wavelength band ranging from 350 nm to 400 nm, more preferably, light having a wavelength band ranging from 370 nm to 390 nm. Light emitted by the first light-emitting diode group 411 is directly emitted to the fluid sucked into the outer case 100 to remove various microorganisms in the fluid through sterilization. Accordingly, the fluid having been subjected to the sterilization due to the first light-emitting diode group 411 is introduced into the fluid cleaning filter 200. Also, remaining part of the light emitted by the first light-emitting diode group 411 is directly or indirectly emitted to the fluid cleaning filter 200 to activate the photocatalyst 300 coated on the surface of the fluid cleaning filter 200. Due to the activation of the photocatalyst 300, the contaminants such as organic materials including various disease-causing germs and bacteria adsorbed onto the fluid cleaning filter 200 are photooxidative-decomposed.

The light having the second wavelength band may be deep ultraviolet light having a wavelength band ranging from 200 nm to 300 nm. More preferably, the light having the second wavelength band may be light having a wavelength band ranging from 250 nm to 290 nm. Accordingly, the second light-emitting diode group 412 may include one or more light-emitting diodes emitting light having a wavelength band ranging from 200 nm to 300 nm, more preferably, light having a wavelength band ranging from 250 nm to 290 nm. In particular, the light having the second wavelength band emitted by the second light-emitting diode group 412 is directly emitted to the fluid introduced into the outer case 100 to remove various microorganisms in the fluid through sterilization. In particular, since the light having the second wavelength band emitted by the second light-emitting diode group 412 has much higher sterilization power that destroys cell walls or cell membranes of microorganisms than the light having the first wavelength band, the fluid to be cleaned may be firstly pre-sterilized and then may be introduced into the fluid cleaning filter 200.

As described above, since the light source unit 400 includes the first and second light-emitting diode groups 411 and 412, the fluid firstly sterilized may be introduced into the fluid cleaning filter 200.

The apparatus may further include the fan 500. The fan 500 is mounted on the rear surface of the fluid cleaning filter 200 by a fan fixing unit 700, and ejects the fluid cleaned by the fluid cleaning filter 200 to the outside using a rotational force. Accordingly, the fan 500 may be disposed to face the rear surface of the fluid cleaning filter 200 in the direction in which the fluid is introduced. That is, the fan 500 is disposed behind the fluid cleaning filter 200 in the direction in which the fluid is introduced such that the rotation surface faces the rear surface of the fluid cleaning filter 200.

The apparatus may further include the pre-filter 600. The pre-filter 600 is received in the pre-filter receiving unit 140 of the outer case 100, and functions to filter particles having relatively large sizes before the fluid sucked into the outer case 100 is sterilized by the light source unit 400. Accordingly, the pre-filter 600 may be disposed in front of the fluid cleaning filter 200 in the direction in which the fluid is introduced, and may be received in the outer case 100 such that the filtering surface faces the front surface of the fluid cleaning filter 200.

While the invention has been shown and described with reference to certain exemplary embodiments thereof, it will be understood by those skilled in the art that various changes in form and details may be made therein without departing from the spirit and scope of the invention as defined by the appended claims.

### DESCRIPTION OF REFERENCE NUMERALS

100: outer case
110: filter receiving unit
121: first light source mounting unit
125: second light source mounting unit
130: fan mounting unit
140: pre-filter receiving unit
150: filter mounting unit
200: fluid cleaning filter
300: photocatalyst
400: first light source unit
450: second light source unit
410: light-emitting unit
411: first light-emitting diode group
412: second light-emitting diode group
420: fixing unit
500: fan
600: pre-filter
700: fan fixing unit

1. An apparatus for cleaning a fluid, comprising:
an outer case;
a fluid cleaning filter disposed in the outer case to filter the fluid introduced;
a photocatalyst coated on a surface of the fluid cleaning filter; and
a first light source unit mounted on the outer case to emit light having a plurality of wavelength bands toward a photocatalyst-coated surface of the fluid cleaning filter.

2. The apparatus of claim 1, wherein the first light source unit is mounted on the outer case to face a filtering surface of the fluid cleaning filter in a direction in which the fluid is introduced, and emits light toward the filtering surface of the fluid cleaning filter.

3. The apparatus of claim 1, wherein the first light source unit comprises an ultraviolet light-emitting diode.

4. The apparatus of claim 1, wherein the first light source unit comprises:
a first light-emitting diode group that emits light having a first wavelength band; and
a second light-emitting diode group that emits light having a second wavelength band.

5. The apparatus of claim 4, wherein the first light-emitting diode group comprises one or more light-emitting diodes emitting light having a wavelength band ranging from 350 nm to 400 nm, and the second light-emitting diode group comprises one or more light-emitting diodes emitting light having a wavelength band ranging from 200 nm to 300 nm.

6. The apparatus of claim 4, wherein the first light-emitting diode group comprises one or more light-emitting diodes emitting light having a wavelength band ranging from 370 nm to 390 nm, and the second light-emitting diode group comprises one or more light-emitting diodes emitting light having a wavelength band ranging from 250 nm to 290 nm.

7. The apparatus of claim 1, wherein the fluid cleaning filter is formed of a metal foam.

8. The apparatus of claim 1, wherein the photocatalyst is coated on a front surface or a rear surface of the fluid cleaning filter in a direction in which the fluid is introduced.

9. The apparatus of claim 1, wherein the photocatalyst comprises any one selected from the group consisting of TiO₂, SrTiO₃, WO₃, ZnO, and a mixture thereof.

10. The apparatus of claim 1, further comprising a fan mounted on the outer case to face a front surface of the fluid cleaning filter in a direction in which the fluid is introduced.

11. The apparatus of claim 10, further comprising a pre-filter disposed between the fan and the fluid cleaning filter.

12. (canceled)

13. The apparatus of claim 1, further comprising a second light source unit disposed in the outer case close to a front surface of the fluid cleaning filter in a direction in which the fluid is introduced, and emits light having a plurality of wavelength bands toward the fluid introduced toward the fluid cleaning filter.

14. The apparatus of claim 13, further comprising at least one partition wall unit disposed in the outer case to face the front surface of the fluid cleaning filter in the direction in which the fluid is introduced.

15. The apparatus of claim 13, wherein the second light source unit is disposed along a circumference of the outer case close to the front surface of the fluid cleaning filter.

16. The apparatus of claim 14, wherein the second light source unit is disposed on a front surface and/or a rear surface of the partition wall unit in the direction in which the fluid is introduced, and/or is disposed along a circumference of the outer case close to the front surface of the fluid cleaning filter.

17. The apparatus of claim 13, wherein the second light source unit comprises:
a first light-emitting diode group that comprises one or more light-emitting diodes emitting light having a wavelength band ranging from 350 nm to 400 nm; and
a second light-emitting diode group that comprises one or more light-emitting diodes emitting light having a wavelength band ranging from 200 nm to 300 nm.

18. The apparatus of claim 17, wherein the first light-emitting diode group comprises one or more light-emitting diode emitting light having a wavelength band ranging from 370 nm to 390 nm, and the second light-emitting diode group comprises one or more light-emitting diodes emitting light having a wavelength band ranging from 250 nm to 290 nm.

19. The apparatus of claim 13, further comprising a photocatalyst coated on the front surface or a rear surface of the fluid cleaning filter in the direction in which the fluid is introduced,
wherein the photocatalyst is any one selected from the group consisting of TiO₂, SrTiO₃, WO₃, ZnO, and a mixture thereof.

20. The apparatus of claim 13, further comprising a fan mounted on a rear surface of the fluid cleaning filter in the direction in which the fluid is introduced.

21. The apparatus of claim 20, further comprising a pre-filter mounted on the outer case to face the front surface of the fluid cleaning filter in the direction in which the fluid is introduced.
